(19) United States
(12) Patent Application Publication
(10) Pub. No.: US 2009/0136463 A1
(43) Pub. Date: May 28, 2009

(54) Schwann Cell Bridge Implants and Phosphodiesterase Inhibitors to Stimulate CNS Nerve Regeneration

(75) Inventors: Mary Bartlett Bunge, Miami, FL (US); Damien Daniel Pearse, Miami, FL (US)

Correspondence Address: VENABLE LLP, P.O. BOX 34385, WASHINGTON, DC, 20043-9998, US

(73) Assignee: University of Miami, Miami, FL (US)

(21) Appl. No.: 12/363,388

(22) Filed: Jan. 30, 2009

Related U.S. Application Data
(63) Continuation of application No. 10/359,554, filed on Feb. 7, 2003.
(60) Provisional application No. 60/354,306, filed on Feb. 7, 2002.

(51) Int. Cl. A61K 35/12

(52) U.S. Cl. 424/93.7

(57) ABSTRACT

The use of a composition that elevates intracellular levels of cyclic nucleotide cyclases in combination with phosphodiesterase inhibitors and cell grafts to restore function after CNS injury.

This application claims priority to U.S. provisional application no. 60/354,306, filed Feb. 7, 2002, which is incorporated herein by reference in its entirety. The invention was developed in part with funds from NIH Grant Nos. NINDS 09923 and POINS 38665. The U.S. Government has certain rights in the invention.

### BACKGROUND OF THE INVENTION

1. Technical Field

This invention relates to the use of cyclic nucleotide cyclases and their activators in combination with phosphodiesterase inhibitors and cell grafts to restore function after central nervous system (CNS) injury.

2. Background Information

The lack of axonal regeneration in the injured or diseased adult mammalian CNS leads to permanent functional impairment. Spinal cord injury alone, for example, affects more than 250,000 people in the U.S. Whereas injured axons in the peripheral nervous system (PNS) successfully regrow and reestablish contacts with denervated targets, axonal regeneration in the CNS is abortive, leading to permanent loss of functions. The failure of CNS axons to regenerate has been related in part to the nonpermissive nature of the glial environment surrounding the injury site or area of lost or damaged tissue.

Schwann cells (SC) have been shown to promote regeneration in both the peripheral (Rodriguez et al., 2000) and central nervous systems, in both the spinal cord (Xu et al., 1997) and brain (Brook et al., 2001; Collier et al., 1999) after both injury and disease. When SC-seeded guidance channels are grafted into transected spinal cords or nerves in animal models, axonal regeneration is enhanced, indicating promise that this or similar techniques may improve or restore function when further developed and refined. One promising area of research has been the addition of trophic factors and other agents that may act at the cellular level to directly stimulate axonal growth, or to counteract inhibitory substances that may be present at the site of the injury. Despite intensive research over the last several decades, however, effective treatment for CNS injuries have been elusive. Accordingly, there remains a compelling need for new effective treatments for CNS injury and the associated functional impairment.

### SUMMARY

This invention provides a new therapeutic strategy to promote growth of regenerated axons into and from a cell graft placed into the injured CNS. It has been discovered, unexpectedly, that if a composition that elevates intracellular levels of a cyclic nucleotide cyclase (such as, for example, cAMP, cGMP, dibutyryl-cAMP), is administered along with a phosphodiesterase inhibitor (such as, for example, rolipram), to an animal into which cells that provide or mimic functions of neural cells native to the animal's nervous system have been transplanted, a marked improvement in function (consistent stepping, consistent coordination and correct foot placement and the ability to perform fine motor tasks in a similar fashion to the uninjured animal) is seen. Such improvement is not observed in animals receiving a cell graft alone with a cyclic nucleotide cyclase-elevating compound.

Accordingly, this invention provides methods of restoring motor and/or sensory function to an animal following CNS injury. In the methods described herein, cells that provide or mimic the functions of neural cells native to the animal's nervous system are implanted at the site of CNS injury and both a cyclic nucleotide phosphodiesterase inhibitor and a composition that elevates intracellular levels of a cyclic nucleotide cyclase are administered to the animal. The implanted cells can be derived autologously, heterologously or xenologously.

The phosphodiesterase (PD) inhibitor (e.g. rolipram) may be administered prior to, or simultaneously with a composition that elevates intracellular levels of a cyclic nucleotide cyclase and is preferably delivered continuously until it is deemed by the skilled practitioner that further gain of function is unlikely. The PD inhibitor may be administered systemically or to the area of the injury. In many cases, it will be preferable to administer the PD inhibitor locally to the area of the injury, for example using a minipump, so that larger concentrations of the inhibitor can be delivered to the injured area while minimizing any systemic side effects to the animal. In a preferred embodiment, the PD inhibitor is rolipram administered at an dosage of between 0.5 mg/kg and 200 mg/kg per day. Effective dosages of rolipram or other phosphodiesterase inhibitors for individual circumstances can be determined by persons of skill in the art without undue experimentation.

The composition that elevates intracellular levels of a cyclic nucleotide cyclase can include either a cyclic nucleotide cyclase activator or a stable form of cAMP or cGMP. The composition that elevates intracellular levels of a cyclic nucleotide cyclase is preferably administered to the area of the injury or to the damaged neurons whose axonal passage is affected by the injury. The composition preferably includes dibutyryl-cAMP administered in a dosage between 1 mg and 1000 mg per single administration. Effective dosages of db-cAMP or other cyclic nucleotide activators for individual circumstances can be determined by the skilled practitioner without undue experimentation.

Cells that provide or mimic the functions of neural cells native to the animal's nervous system (e.g., Schwann cells) are also introduced into the area of injury, either by injection or by transplantation into a complete transection gap. The cells to be injected or transplanted may be an autograft, homograft, allograft or xenograft. Preferably the cells are autologous.

In a preferred embodiment, the methods of the present invention are used in humans. However, they are considered to be suitable for mammals generally, and should be useful for nonmammalian species having central nervous systems biochemical/physiological/anatomical characteristics and features similar to humans.

The invention also includes a pharmaceutical composition comprising a phosphodiesterase inhibitor and a compound that elevates intracellular levels of a cyclic nucleotide cyclase, for example rolipram and db-cAMP, as well as a composition comprising a phosphodiesterase inhibitor, a compound that elevates intracellular levels of a cyclic nucleotide cyclase, and cells having neural function.

### BRIEF DESCRIPTION OF THE DRAWINGS

FIG. 1 compares the effects over time of db-cAMP injection and rolipram on BBB scores of rats receiving a Schwann cell bridge after complete transection of the spinal cord. Diamonds (control): saline injection into both spinal cord stumps; squares: 0.2 μL×1 mM db-cAMP administered by injection into both spinal cord stumps; triangles: 0.2 μL×25 mM db-cAMP: crosses: 0.2 μL×50 mM camp; circles: 0.2 μL×1 mM db-cAMP and rolipram administration by minipump (0.07 μmol/kg/hr for 2 weeks after injury).

FIG. 2 compares the effects over time of dh-cAMP superfusion and rolipram on BBB scores of rats receiving a Schwann cell bridge after complete transection of the spinal cord, Diamonds (control): saline infusion by a biomaterial (gelfoam) into both spinal cord stumps; squares: 5 μL×1 mM cAMP administered by infusion into both spinal cord stumps; triangles: 5 μL×5 mM cAMP administered by infusion into both spinal cord stumps; crosses: 5 μL×10 mM cAMP administered by infusion into both spinal cord stumps; circles: 5 μL×1 mM cAMP administered by infusion into both spinal cord stumps and rolipram administration by minipump (0.07 μmol/kg/hr for 2 weeks after injury).

FIG. 3 compares the effects over time of db-cAMP injection and rolipram on BBB scores of rats receiving Schwann cell transplantation after receiving moderate contusion injury to the spinal cord by weight drop (NYU impactor, 12.5 mm height). Each treatment used 4 injection sites, 2-3 mm rostral and caudal to the injury site and on either side of the midline. Diamonds: 2×10⁶ Schwann cells were injected into the injury site with saline injection one week post-injury; squares: 2×10⁶ Schwann cells with 0.2 μL×1 mM db-cAMP (four injections) one week post-injury; triangles: 2×10⁶ Schwann cells with 0.2 μL×50 mM db-cAMP (four injections) one week post-injury; crosses: 2×10⁶ Schwann cells with 0.2 μL×50 mM cAMP (four injections) one day post-injury; circles: animals received rolipram by minipump starting within 30 minutes of the injury (0.07 μmol/kg/hr for 2 weeks after injury) and 1 week later Schwann cells with 0.2 μL×50 mM db-cAMP (four injections).

FIG. 4 compares footfall errors in a gridwalking analysis 8 weeks after a moderate contusion injury by weight drop (NYU impactor, 12.5 mm height) followed by db-cAMP administration plus Schwann cell transplantation with or without rolipram. Each treatment used 4 injection sites, 2-3 mm rostral and caudal to the injury site and on either side of the midline. A. Non-injured control; B. 1 week post-injury 2×10⁶ Schwann cells injected into the injury site with saline; C. 1 week post-injury 2×10⁶ Schwann cells injected with 4×0.2 μL×1 mM db-cAMP; D. 1 week post-injury 2×10⁶ Schwann cells with 4×0.2 μL×50 mM db-cAMP; E. 1 day post-injury 2×10⁶ Schwann cells with 4×0.2 μL×50 mM db-cAMP; F. animals received rolipram by minipump starting within 30 minutes of the injury (0.07 μmol/kg/hr for 2 weeks after injury) and 1 week later 2×10⁶ Schwann cells with 4×0.2 μL×50 mM db-cAMP.

FIG. 5 compares stride length in a footprint analysis conducted 8 weeks after a moderate contusion injury by weight drop followed by db-cAMP administration plus Schwann cell transplantation with or without rolipram. Each treatment used 4 injection sites, 2-3 mm rostral and caudal to the injury site and on either side of the midline. A. Non-injured control; B. 1 week post-injury 2×10⁶ Schwann cells injected into the injury site with saline; C. 1 week post-injury 2×10⁶ Schwann cells injected with 4×0.2 μL×1 mM db-cAMP: D. 1 week post-injury 2×10⁶ Schwann cells with 4×0.2 μL×50 mM: db-cAMP; E. 1 day post-injury 2×10⁶ Schwann cells with 4×0.2 μL×50 mM db-cAMP; F. animals received rolipram by minipump starting within 30 minutes of the injury (0.07 μmol/kg/hr for 2 weeks after injury) and 1 week later 2×10⁶ Schwann cells with 4×0.2 μL×50 mM: db-cAMP.

FIG. 6 compares base of support in a footprint analysis conducted 8 weeks after a moderate contusion injury by weight drop followed by db-cAMP administration plus Schwann cell transplantation with or without rolipram, Each treatment used 4 injection sites, 2-3 mm rostral and caudal to the injury site and on either side of the midline. A. Non-injured control; B. 1 week post-injury 2×10⁶ Schwann cells injected into the injury site with saline; C. 1 week post-injury 2×10⁶ Schwann cells injected with 4×0.2 μL×1 mM: db-cAMP; D. 1 week post-injury 2×10⁶ Schwann cells with 4×0.2 μL×50 mM db-cAMP; E. 1 day post-injury 2×10⁶ Schwann cells with 4×0.2 μL×50 mM db-cAMP: F. animals received rolipram by minipump starting within 30 minutes of the injury (0.07 μmol/kg/hr for 2 weeks after injury) and 1 week later 2×10⁶ Schwann cells with 4×0.2 μL×50 mM db-cAMP.

FIG. 7 compares angle of foot exo-rotation in a footprint analysis conducted 8 weeks after a moderate contusion injury by weight drop followed by db-cAMP administration plus Schwann cell transplantation with or without rolipram. Each treatment used 4 injection sites, 2-3 mm rostral and caudal to the injury site and on either side of the midline. A. Non-injured control; B. 1 week post-injury 2×10⁶ Schwann cells injected into the injury site with saline; C. 1 week post-injury 2×10⁶ Schwann cells injected with 4×0.2 μL×1 mM db-cAMP; D. 1 week post-injury 2×10⁶ Schwann cells with 4×0.2 μL×50 mM db-cAMP; E. 1 day post-injury 2×10⁶ Schwann cells with 4×0.2 μL×50 mM db-cAMP; F. animals received rolipram by minipump starting within 30 minutes of the injury (0.07 μmol/kg/hr for 2 weeks after injury) and 1 week later 2×10⁶ Schwann cells with 4×0.2 μL×50 mM db-cAMP.

### DETAILED DESCRIPTION OF THE INVENTION

In one aspect, the invention provides a method of treating an animal following injury to an area of the animal's central nervous system that comprises

a) administering a cyclic nucleotide phosphodiesterase inhibitor to the animal;

b) administering a composition that elevates intracellular levels of a cyclic nucleotide cyclase to the animal; and

c) implanting cells that provide or mimic the functions of neural cells native to the animal's nervous system,

so that motor and/or sensory function is improved or restored in the animal.

By “improvement of or restoration of function” is meant a statistically significant improvement in motor or sensory function as measured by the BBB test or other measurements accepted in the field. There are other tests, such as grid walking and footprint analysis, but due to its ease and execution, the BBB test has become the most popular mode of evaluation of hindlimb locomotion. However, the methods described herein will be applicable to many other situations in the central nervous system in which the regrowth of nerve fibers would be helpful in improving lost function and numerous tests exist to analyse the spectrum of functional deficits associated with these.

The composition that elevates intracellular levels of a cyclic nucleotide cyclase can include either a cyclic nucleotide cyclase activator or a stable form of cAMP or cGMP that can be taken up into cells or a phosphodiesterase-resistant form of a cyclic nucleotide cyclase or phosphodiesterase-resistant activator of a cyclic nucleotide cyclase-dependent protein kinase (for example, analogues of 1-beta-D-ribofuranosylbenzimidazole 3′,5′-phosphate [cBIMP], as described in Genieser et al., 1992). Suitable activators of a cyclic nucleotide cyclase for use in the invention are intended to include any agent capable of elevating intracellular levels of cAMP and/or cGMP, for example forskolin, 7β-deaceyl-7β-[γ(morpholino)butyryl]-forskolin, and 6β-[β′-(piperidino)-propionyl]-forskolin. Stable forms of cAMP and/or cGMP include dibutyryl-cAMP, 8-bromo-adenosine 3′,5′-monophosphate (8-Br-cAMP), 8-(4-chlorophenylthio)-cAMP, 8-chloro-adenosine 3′,5′-monophosphate (8-Cl-cAMP), dioctanoyl-cAMP, Sp-cAMPS, Sp-8-bromo-cAMPS, 8-br-cGMP, dibutyryl-cGMP and 8-(4-chlorophenylthio)-cGMP. Novel activators can be designed by employing in vitro assays to screen prospective compounds for their ability to activate either adenylate or guanylate cyclase, using screening techniques known in the art.

Suitable phosphodiesterase inhibitors are intended to include any cyclic nucleotide phosphodiesterase inhibitor that may be administered systemically or locally to a mammal without causing adverse effects that would be considered unacceptable by persons of skill in the art. It will be appreciated that any such adverse effects must be balanced against the benefits of the treatment of the invention, i.e. an improvement or restoration of motor function following paralysis or other consequences of nerve damage to the spinal cord. Suitable phosphodiesterase inhibitors include, inter alia, 4-(3-cyclopentyloxy-4-methoxyphenyl)-2-pyrrolidone (rolipram), 3-isobutyl-1-methylxanthine (IBMX), 2-(2-propyloxyphenyl)-8-azapurin-6-one (zaprinast), N-(3,5-dichlorpyrid-4-yl)-3-cyclopentyl-oxy-4-methoxy-benzamide (RPR-73401), 8-methoxy-5-N-propyl-3-methyl-1-ethyl-imidazo[1,5-a]-pyrido[3,2-e]-pyrazinone (D-22888), methyl-2-(4-aminophenyl)-1,2-dihydro-1-oxo-7-(2-pyridinylmethoxy)-4-(3,4,5-trimethoxyphenyl)-3-isoquinoline carboxylate sulfate (T-1032), 4-(3-butoxy-4-methoxybenzyl)-2-imidazolidinone (Ro-20-1724), 4-(3-chlorophenyl)-1,7-diethylpyrido[2,3-d]pyrimidin-2(1H)-one (YM976), N-cyclohexyl′N-methyl-4-(1,2-dihydro-2-oxo-6-quinolyloxy) butyramide (cilostamide), dipyridamole, milrinone, amrinone, olprinone, pentoxifylline, theophylline, cilostazol, sildenafil, nimesulide and antisense sequences or vectors designed to be complementary to, and prevent the processing of, the mRNA of a cyclic nucleotide phosphodiesterase. Novel agents can be designed by employing in vitro assays to screen prospective compounds for their ability to inhibit either cAMP or cGMP phosphodiesterases. Persons of skill in the art are familiar with means of obtaining suitable antisense vectors (e.g. Mautino and Morgan, 2002; Pachori et al., 2002).

Suitable transplanted cells are intended to include any cell type derived autologously or heterologously or xenologously that provide or mimic the functions of those native to the nervous system that may be administered at the site of CNS injury to replace lost tissue within a mammal without causing adverse effects which would be considered unacceptable by persons of skill in the art. It will be appreciated that any such adverse effects must be balanced against the benefits of the treatment, i.e. an improvement or restoration of motor function following paralysis or other consequences of nerve damage to the CNS. Suitable cell types for use in the methods described herein include Schwann cells, neural stem cells, neural precursor cells, neural progenitor cells, neurosphere cells, mesenchymal stem cells, hematopoietic stem cells, glial-restricted precursor cells, embryonic stem cells, bone marrow stromal cells and olfactory ensheathing glia. Novel cell types that are capable of mimicking functions of cells endogenous to the nervous system may be discovered through in vitro analysis of stem cells from all bodily tissues or stem cell lines and used for transplantation.

Spinal cord injury is intended to include transection or contusion of the spinal cord, or any other mechanical injury to the spinal cord that results in a measurable loss of function, particularly in motor function. Brain injury is intended to include any mechanical trauma to the brain or detrimental physiological occurrence that results in damage to neurons and/or axons and produces a measurable loss of function. CNS disease is intended to include any abnormal state of the CNS that has resulted in neuron and/or axonal loss or disruption and an accompanying measurable functional loss.

The PD inhibitor and cyclic nucleotide cyclase-affecting composition may be administered systemically or applied locally in the area of the injury. This will usually mean within 2-3 cm of the location of the contusion or transection or cell loss or axon disruption, although greater distances from the injury site may be necessary in some cases where axonal transport is inadequate. The administration procedure would involve the administration of said compounds near to the cell body of the damaged neuron to facilitate uptake and activation of regeneration programs that would produce axon growth. One of the goals of developing a therapeutic strategy is that it would be easily administered to an injured person as soon as possible after injury. This means that it should be a very easy task to administer the therapeutic agent, such as simple subcutaneous injection. An advantage of rolipram is that this can be injected in this manner. Numerous techniques however are available for promoting the delivery of compounds to the CNS. These include, but are not limited to, direct injection or infusion in osmotic minipumps, inclusion within or upon implanted biomaterials (eg. collagen, as fibers, rods or microspheres), by tablet or microcapsule or expressed in genetically transformed grafted cells as antisense vectors or in the form of genes that are activators of cyclic nucleotide cyclases.

The transplanted cells are administered locally to the injury, either by injection, or by implantation of a cell bridge, as detailed below. The transplanted cells are positioned at the site of spinal cord transection, contusion, or cell loss or the site of injury or cell loss or axon damage in the injured or diseased brain. The cells are preferably genetically similar to the individual receiving the graft, although cells that originate from another individual of the same species, or in some instances from a different species may be acceptable. One of the advantages of using Schwann cells for implantation is that they can be prepared from the person who is to receive the implant. That is, they can be autotransplanted. From a piece of peripheral nerve removed from an injured person, the technology is now available to expand a small number of cells within a few weeks to a far larger number of cells, enabling the preparation of a graft that is half an inch in diameter, and perhaps as much as 1 meter long. While the Schwann cells are multiplying in culture, they can also be genetically engineered to produce higher amounts of certain growth factors that, are known to promote nerve fiber regrowth (see, for example, Blits et al., 1999, Blits et al., 2000). Millions of Schwann cells can be injected in a very small volume, 0.4 μl, for example, into a mammalian spinal cord by means of a syringe. It should be especially noted that techniques are currently available to create large numbers of human, as well as rat, Schwann cells. Production of such cells from other animals is expected to be routine.

The phosphodiesterase inhibitor is preferably administered prior to, but can be administered simultaneously with, the composition that elevates intracellular levels of a cyclic nucleotide cyclase and cell grafting. Administration of the phosphodiesterase inhibitor must be maintained during and after administration of the composition that elevates intracellular levels of a cyclic nucleotide cyclase and cell grafting. The phosphodiesterase inhibitor can be administered continuously over a long period of time (e.g. hours, days, weeks or longer) by use of an osmotic minipump (such as those manufactured by DURECT Corporation, Cupertino, Calif.), by repetitive systemic injection, by biomaterials (implanted within the individual where the agent is embedded within or coated upon eg. collagen, as fibers, rods or microspheres), or by formulation in a tablet or microcapsule to be given repeatedly by oral administration. The phosphodiesterase inhibitor may also be contained within transformed grafted cells in the form of a phosphodiesterase antisense vector or as an antisense oligonucleotide that is complementary to the mRNA of a cyclic nucleotide

Dosages of phosphodiesterase inhibitor and the cyclic nucleotide cyclase activator or stable form of cAMP or cGMP can be determined empirically by the skilled practitioner, and will depend upon the specific phosphodiesterase inhibitor and the cyclic nucleotide cyclase activator or stable form of cAMP or cGMP, the formulation, the route of administration, the individual, type and severity of injury, and other circumstances of the ease etc. In general, 1 mg to 1,000 mg of db-cAMP or another cyclic nucleotide cyclase activator or stable form of cAMP or cGMP will be delivered to the site of the injury at the time of cell implantation or afterwards; rolipram or another phosphodiesterase inhibitor will be administered continuously before cell grafting, as soon as possible after injury, at a rate of between 0.5 mg/kg and 200 mg/kg daily for a period encompassing the time of the cyclic nucleotide cyclase activator, or stable form of cAMP or cGMP, administration and cell grafting, and during subsequent recovery until it is deemed by the skilled practitioner that further gain of function is unlikely.

It is believed that the methods described herein will function best when treatment begins as soon following injury. Although benefit can be expected for any length of time following injury, greatest restoration of function is expected with rapid intervention.

### EXAMPLE 1

Schwann cells were purified in culture from adult rat sciatic nerve (according to the methods described by Morrissey, Kleitman and Bunge (1991)). The purity of the Schwann cells used for transplantation was between 95 and 98%.

For Schwann cell bridges, cells were suspended in matrigel/DMEM (30:70) and drawn into 3-8 mm long polymer guidance channels at a density of 120×10⁶ cells/ml, as described by Xu et al. (1997). During implantation into adult rats (Fischer rats, Charles River Laboratories, 3-5 months old), each cut stump of the severed spinal cord was inserted 1 mm into the channel. Sometimes the Schwann cell cable is transplanted without the guidance channel. Either method, with or without the channel, is readily accomplished by persons who have performed this procedure a number of times and so have gained adequate expertise to accomplish this.

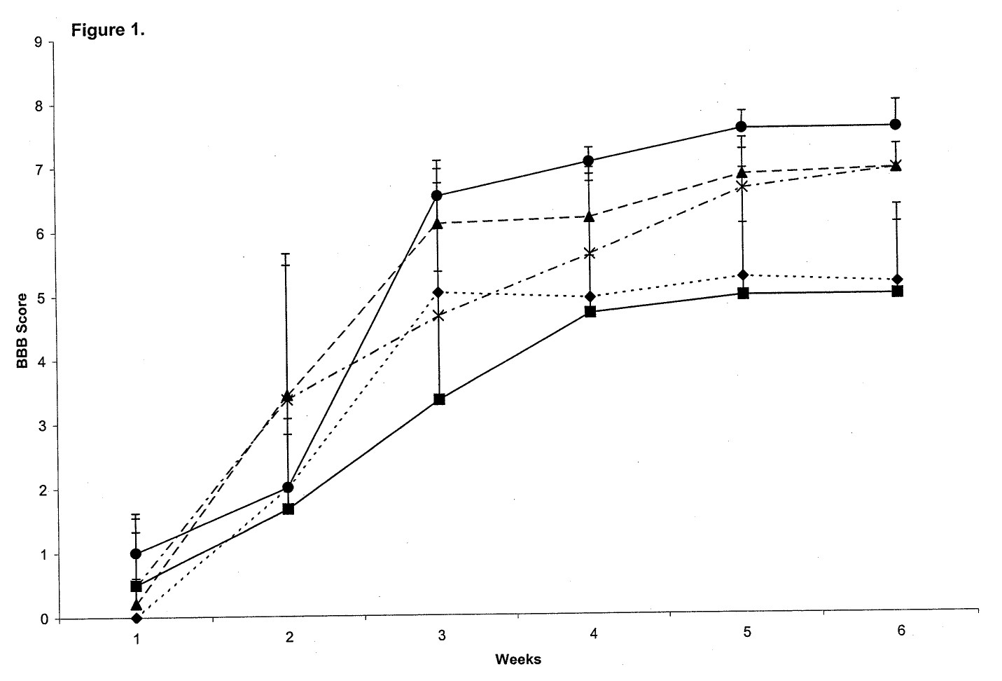

Spinal cords of adult rats were completely transected by surgery at the T8 cord level and the next caudal segment was removed. At the time of transection, a Schwann cell bridge was implanted at the injury site, 50 mM db-cAMP was injected (0.2 μl) or infused (5 μl) into the proximal and distal stump of the lesion and rolipram was delivered subcutaneousiy via minipump at 0.07 μmol/kg/hr for two weeks. One control group received a Schwann cell bridge with saline infusion (5 μl) or injection (0.2 μl) into the proximal and distal stump of the lesion with saline (equivalent volume) delivered also by minipump. The other control groups received 5 μl of 1, 5 or 10 mM db-cAMP, infused into the proximal and distal stump of the lesion or 0.2 μl of 1, 25 or 50 mM db-cAMP, injected into the proximal and distal stump of the lesion with saline delivered by minipump. Animals were assessed on a weekly basis for hindlimb locomotion, a measure of motor recovery, using the BBB test. The results shown in FIG. 1 demonstrate that the combination of a Schwann cell graft with injection of db-cAMP and rolipram facilitates plantar placement without weight support in rats with a complete spinal cord transection at thoracic cord segment 8. This is not observed with db-cAMP or Schwann cell grafts alone or untreated animals.

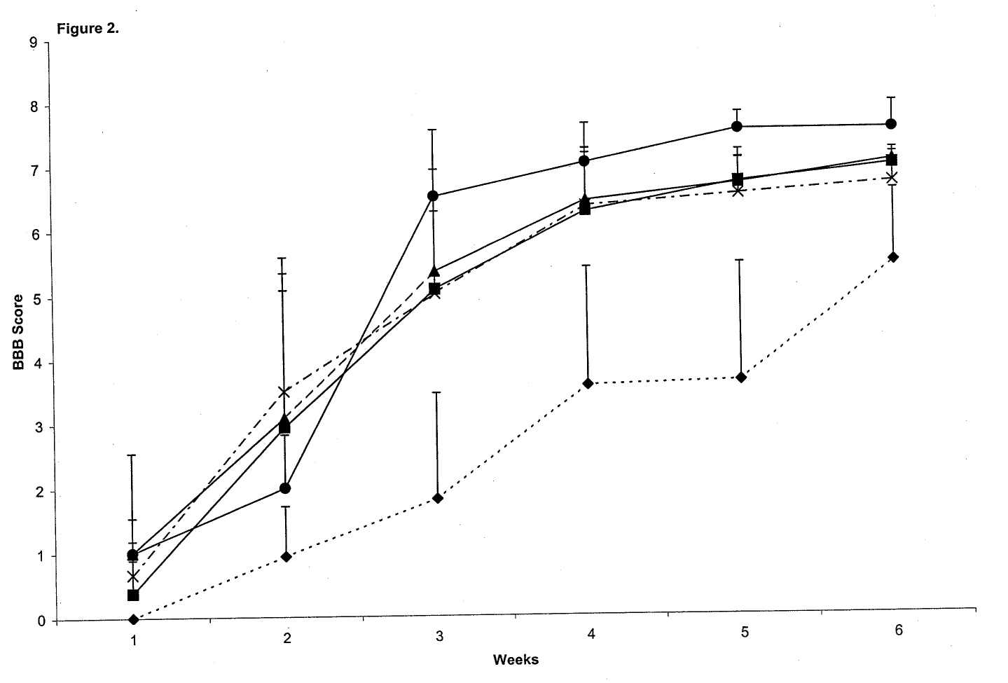

FIG. 2 demonstrates that the combination of a Schwann cell graft, infused db-cAMP and rolipram facilitates plantar placement without weight support in rats with a complete spinal cord transection at thoracic cord segment 8. This is not observed with db-cAMP or Schwann cell grafts alone or untreated animals.

### EXAMPLE 2

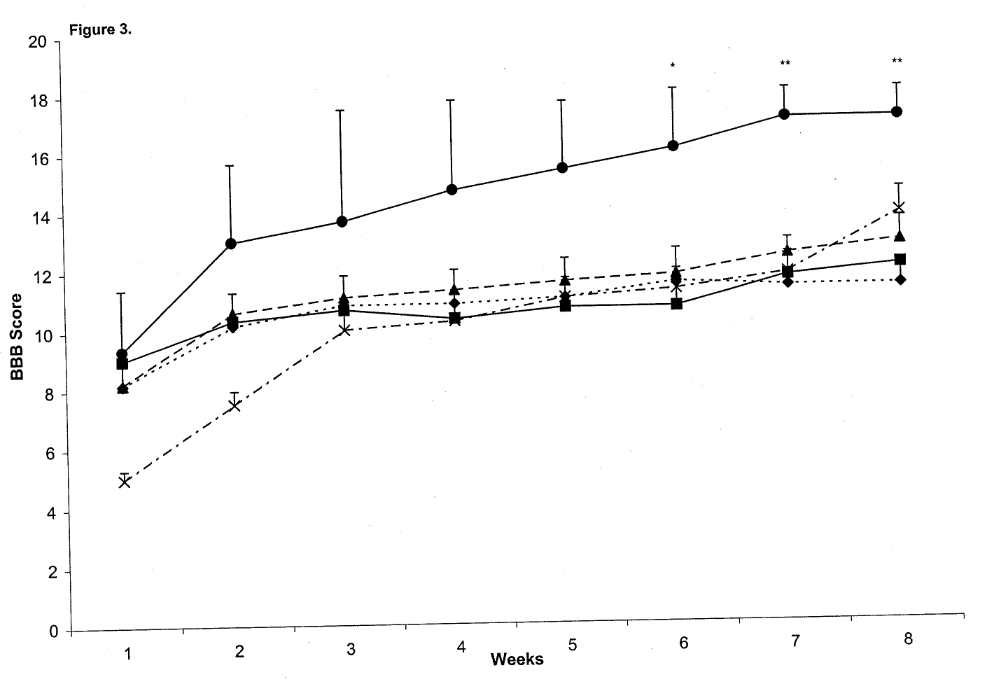

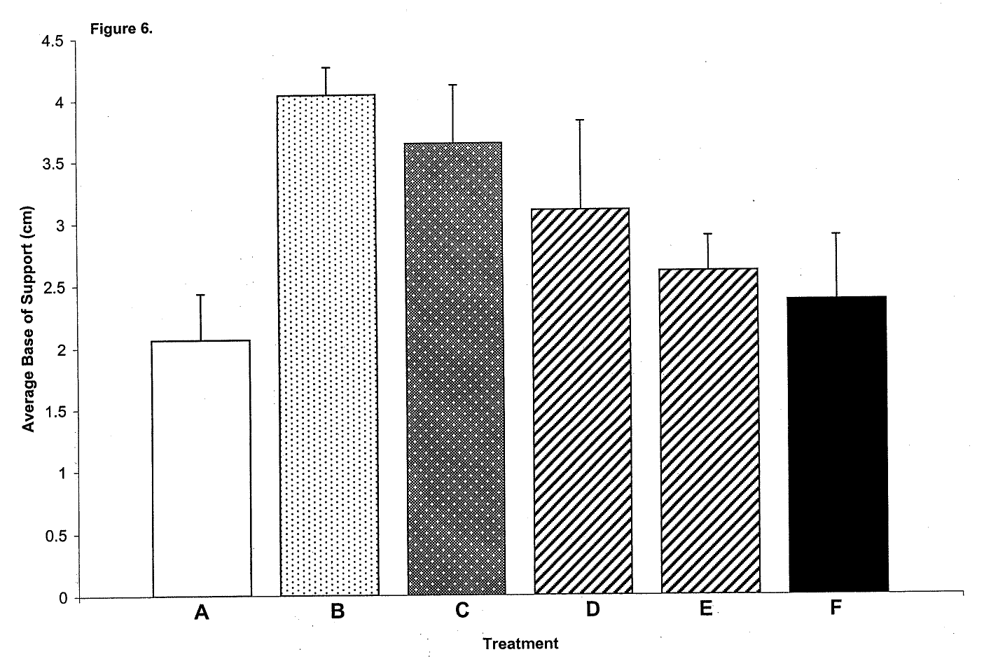

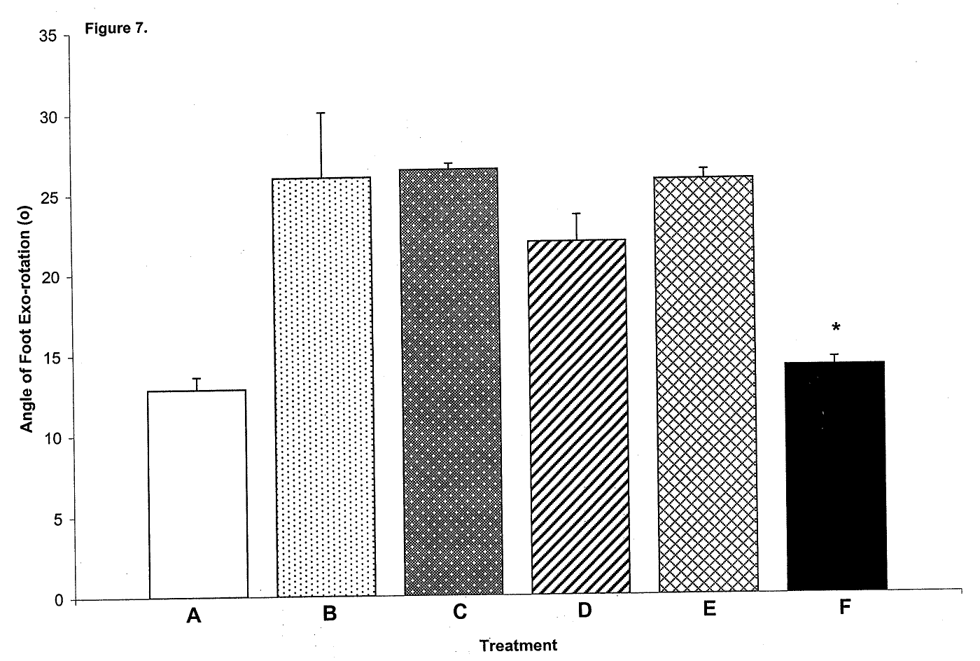

Adult rats (Fischer rats, Charles River Laboratories, 3-5 months old) were injured in the thoracic level of the spinal cord with the NYU weight drop device (NYU impactor) as described in Gruner (1992), and rolipram (0.07 μmol/kg/hr) was administered for two weeks. One day or one week after injury, 2×10⁶ Schwann cells were injected into the lesion site and injections of db-cAMP (1 mM or 50 mM×0.2 μL) were made into either side of the midline just above and below the lesion site. Animals were tested weekly using the BBB test (described in Basso et al.). The gridwalk test for fine locomotor performance and footprint analysis after condition locomotion over a flat surface were used also to examine functional recovery (described in Basso et al.). A marked improvement was seen in the hindlimb locomotion (consistent stepping, consistent coordination, correct foot placement and the ability to perform fine motor tasks at almost the degree of un-injured animals) in those animals that received both the Schwann cell and db-cAMP injections into the cord and rolipram, as compared to animals receiving only dbcAMP or Schwann cells, shown in FIGS. 3-7. FIG. 3 demonstrates that the combination of a Schwann cell graft, injected db-cAMP and rolipram facilitates consistent stepping, consistent coordination and correct foot placement in rats with a moderate contusion injury at thoracic cord segment 8, an improvement that is not observed with db-cAMP or Schwann cell grafts alone or untreated animals.

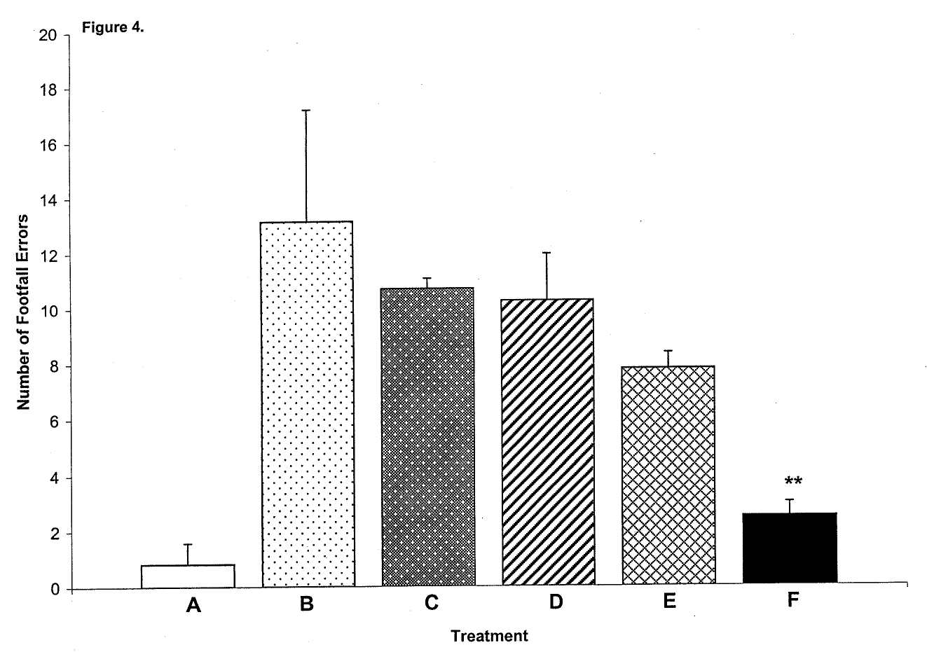

FIG. 4 shows the ability of the injured rats that received various treatments to perform fine motor skills on a 1 m gridwalk apparatus consisting of 10 irregularly spaced bars (separated by 0.5 to 4.5 cm) across which the animals traversed. The number of footfall errors that the animal makes is recorded (maximum is 20, 1 per leg per space between each bar) with higher scores indicating a poor ability to perform the tasks, The results demonstrate that the combination of a Schwann cell graft, injected db-cAMP and rolipram restores the ability to perform fine motor tasks to almost the degree of the un-injured animal in rats with a moderate contusion injury at thoracic cord segment 8. Animals with db-cAMP or Schwann cell grafts alone exhibited many more errors in this task.

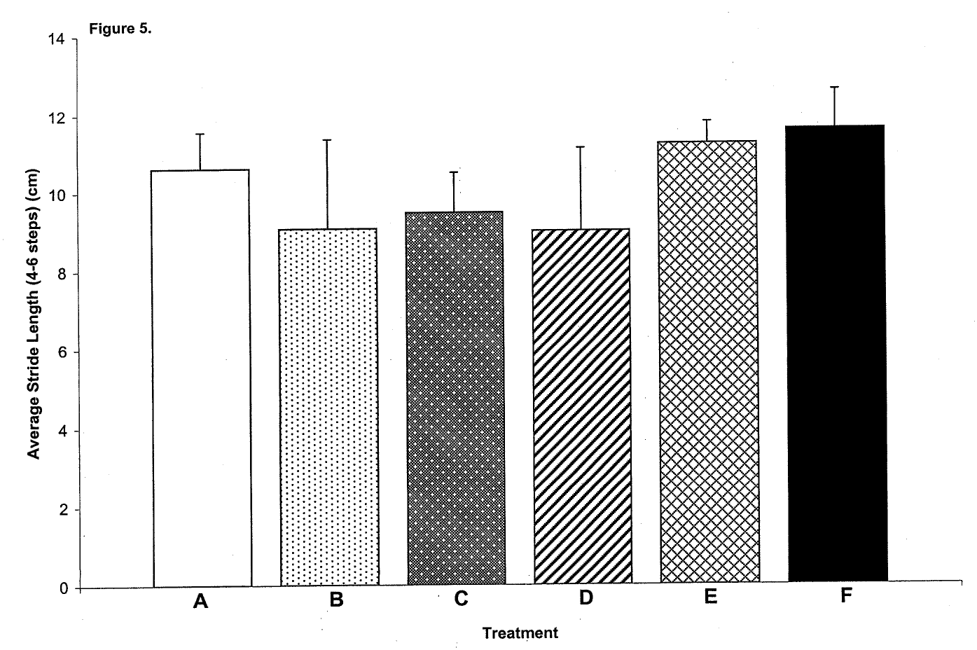

FIGS. 5, 6 and 7 illustrate the locomotor patterns of injured rats that received various treatments, tested by inking both the fore- and hind-paws (different colors), allowing them to walk 1 m on an enclosed, flat runway and then analyzing the footprints. Recorded parameters from 8 consecutive steps included the animal's stride length (measured between the central pads of two consecutive prints on each side of the animal), base of support (determined by measuring the distance between the central pads of the hindpaws), and hindfoot outward rotation. Normal animals exhibit a stride-length of between 10 and 14 cm, that is thought to decrease after SCI, according to the severity of the injury. Base of support is indicative of the trunk stability of the animal. An injured animal will have a larger base of support in order to increase the surface area upon which it is supported to avoid falling over. Outward foot rotation commonly occurs following SCI. A greater angle of foot rotation is observed according to the severity of the injury. The FIG. illustrates the ability of non-injured rats and compares 1) control rats that received a moderate contusion injury by weight drop (NYU impactor, 12.5 mm height) and which received 1 week later 2×10⁶ Schwann cells injected into the injury site with saline injection (4 injection sites, 2-3 mm rostral and caudal to the injury site and on either side of the midline), 2) rats that received 1 week after contusion Schwann cells with 1 mM cAMP (4 injection sites, 2-3 mm rostral and caudal to the injury site and on either side of the midline), 3) or rats that received 1 week after contusion Schwann cells with 50 mM cAMP, 4) rats that received 1 day after contusion Schwann cells with 50 mM cAMP, 5) as in 3 but that received rolipram by minipump starting within 30 minutes of the injury (0.07 μmol/kg/hr for 2 weeks after injury) and 1 week later Schwann cells with 50 mM cAMP.

FIGS. 5, 6 and 7 demonstrate that the combination of a Schwann cell graft, injected db-cAMP and rolipram restores trunk instability and reduces outward foot rotation during conditioned locomotion in rats with a moderate contusion injury (weight drop 12.5, NYU device) at thoracic cord segment 8. Animals with db-cAMP or Schwann cell grafts alone did not exhibit a similar level of recovery.

References cited are listed below for convenience and are hereby incorporated by reference.

### REFERENCES

Basso D M, Beattie M S, Bresnahan J C (1995) A sensitive and reliable locomotor rating scale for open field testing in rats, J Neurotrauma 12: 1-21.
Blits B, Dijkhuizen P A, Carlstedt T P, Poldervaart H, Schiemanck S, Boer G J, Verhaagen J (1999) Adenoviral vector-mediated expression of a foreign gene in peripheral nerve tissue bridges implanted in the injured peripheral and central nervous system Exp Neurol 160:256-67.
Blits B, Dijkhuizen P A, Boer G J, Verhaagen J (2000) Intercostal nerve implants transduced with an adenoviral vector encoding neurotrophin-3 promote regrowth of injured rat corticospinal tract fibers and improve hindlimb function Exp Neurol 164:25-37.
Brook G A, Lawrence J M, Raisman G (2001) Columns of Schwann cells extruded into the CNS induce in-growth of astrocytes to form organized new glial pathways, Glia 33:118-130.
Chen A, Xu X M, Kleitman, N, Bunge M B (1996) Methylprednisolone administration improves axonal regeneration into Schwann cell grafts in transected adult rat thoracic spinal cord.
Collier T J, Sortwell C E, Daley B F (1999) Diminished viability, growth, and behavioral efficacy of fetal dopamine neuron grafts in aging rats with long-term dopamine depletion: an argument for neurotrophic supplementation, J Neurosci 19:5563-5573.
Genieser H G, Winkler E, Butt E, Zorn M, Schulz S, Iwitzki F, Stormann R, Jastorff B, Doskeland S O, Ogreid D, et al. Derivatives of 1-beta-D-ribofuranosylbenzimidazole 3′,5′-phosphate that mimic the actions of adenosine 3′,5′-phosphate (cAMP) and guanosine 3′,5′-phosphate (cGMP). Carbohydr Res 1992 Oct. 9; 234:217-35.
Gruner J A (1992) A monitored contusion model of spinal cord injury in the rat. J Neurotrauma 9:123-126.
Guest, J D, Arundathi, R., Olson, L., Bunge, M B, Bunge, R P (1997) The ability of human Schwann cell grafts to promote regeneration in the transected nude rat spinal cord. Exp Neurology 148:502-521.
Mautino M R, Morgan R A (2002) Enhanced inhibition of human immunodeficiency virus type 1 replication by novel lentiviral vectors expressing human immunodeficiency virus type 1 envelope antisense RNA Hum Gene Ther 13:1027-37.
Morrissey T K, Kleitman N, Bunge R P (1991) Isolation and functional characterization of Schwann cells derived from adult peripheral nerve. J Neurosci 11:2433-2442.
Negishi H, Dezawa M, Oshitari T, Adachi-Usami E (2001) Optic nerve regeneration within artificial Schwann cell graft in the adult rat. Brain Res Bull 55:409-419.
Pachori A S, Numan M T, Ferrario C M, Diz D M, Raizada M K, Katovich M J (2002) Blood pressure-independent attenuation of cardiac hypertrophy by AT(1)R-AS gene therapy, Hypertension 39:969-75.
Rodriguez F J, Verdu E, Ceballos D, Navarro X (2000) Nerve guides seeded with autologous Schwann cells improve nerve regeneration. Exp Neurol 161:571-584.
Xu, X M, Chen, A, Guenard, V, Kleitman, N, Bunge M B (1997) Bridging Schwann cell transplants promote axonal regeneration from both the rostral and caudal stumps of transected adult rat spinal cord. J Neurocytology 26:1-16.

1. A method of treating an animal following injury to an area of the animal's central nervous system, the method comprising:
a) administering a cyclic nucleotide phosphodiesterase inhibitor to the animal;
b) administering a composition that elevates intracellular levels of a cyclic nucleotide cyclase to the animal; and
c) implanting cells that provide or mimic the functions of neural cells native to the animal's nervous system,
so that motor and/or sensory function is improved in the animal.

2. The method of claim 1 wherein the phosphodiesterase inhibitor is administered prior to the composition that elevates intracellular levels of a cyclic nucleotide cyclase.

3. The method of claim 1 wherein the phosphodiesterase inhibitor is administered simultaneously with the composition that elevates intracellular levels of a cyclic nucleotide cyclase.

4. The method of claim 1 wherein the phosphodiesterase inhibitor is administered systemically,

5. The method of claim 1 wherein the phosphodiesterase inhibitor is administered locally in the area of the injury.

6. The method of claim 1 wherein the composition that elevates intracellular levels of a cyclic nucleotide cyclase is administered locally in the area of the injury.

7. The method of claim 1 wherein the step of administering a cyclic nucleotide phosphodiesterase inhibitor comprises administering one or more compounds selected from the group consisting of rolipram, 3-isobutyl-1-methylxanthine (IBMX), 2-(2-propyloxyphenyl)-8-azapurin-6-one (zaprinast), N-(3,5-dichlorpyrid-4-yl)-3-cyclopentyl-oxy-4-methoxy-benzamide (RPR-73401), 8-methoxy-5-N-propyl-3-methyl-1-ethyl-imidazo[1,5-a]-pyrido[3,2-e]-pyrazinone (D-22888), methyl-2-(4-aminophenyl)-1,2-dihydro-1-oxo-7-(2-pyridinylmethoxy)-4-(3,4,5-trimethoxyphenyl)-3-isoquinoline carboxylate sulfate (T-1032), 4-(3-butoxy-4-methoxybenzyl)-2-imidazolidinone (Ro-20-1724), 4-(3-chlorophenyl)-1,7-diethylpyrido[2,3-d]pyrimidin-2(1H)-one (YM976), N-cyclohexyl′N-methyl-4-(1,2-dihydro-2-oxo-6-quinolyloxy) butyramide (cilostamide), dipyridamole, milrinone, amrinone, olprinone, pentoxifylline, theophylline, cilostazol, sildenafil and nimesulide.

8.-10. (canceled)

11. The method of claim 1 wherein the step of administering a composition that elevates intracellular levels of a cyclic nucleotide cyclase comprises administering one or more compounds selected from the group consisting of db-cAMP, 8-bromo-adenosine 3′,5′-monophosphate (8-Br-cAMP), 8-(4-chlorophenylthio)-cAMP, 8-chloro-adenosine 3′,5′-monophosphate (8-Cl-cAMP), dioctanoyl-cAMP, Sp-cAMPS, Sp-8-bromo-cAMPS, 8-br-cGMP, dibutyryl-cGMP and 8-(4-chlorophenylthio)-cGMP.

12. The method of claim 1 wherein the step of administering a composition that elevates intracellular levels of a cyclic nucleotide cyclase comprises administering db-cAMP.

13. The method of claim 12 wherein the dosage of db-cAMP is between 1 mg and 1000 mg per day.

14. The method of claim 1 wherein the step of implanting cells comprises implanting one or more cell types selected from the group consisting of Schwann cells, neural stem cells, neural precursor cells, neural progenitor cells, neurosphere cells, mesenchymal stem cells, hematopoietic stem cells, glial-restricted precursor cells, embryonic stem cells, bone marrow stromal cells and olfactory ensheathing glial cells.

15. The method of claim 1 wherein the step of implanting cells comprises transplanting Schwann cells.

16. The method of claim 15 wherein the step of implanting cells comprises injecting Schwann cells.

17. The method of claim 15 wherein the step of implanting cells comprises implanting a Schwann cell bridge.

18. The method claim 1 wherein the step of implanting cells comprises implanting an autograft.

19. The method of claim 1 wherein the step of implanting cells comprises implanting an allograft.

20. The method of claim 1 wherein the step of implanting cells comprises implanting a homograft.

21.-23. (canceled)

24. A method of treating an animal following injury to a area in the animal's central nervous system, the method comprising:
a) implanting Schwann cells at the site of central nervous system injury;
b) administering rolipram to the animal; and
c) administering dibutyryl-cAMP to the area of the injury during the step of administering rolipram.

25. A pharmaceutical composition comprising an effective amount of a phosphodiesterase inhibitor and a compound that elevates intracellular levels of a cyclic nucleotide cyclase.

26. The composition of claim 25 that additionally comprises an effective amount of cells having neural function.
